(19) United States
(12) Patent Application Publication
(10) Pub. No.: US 2005/0049276 A1
(43) Pub. Date: Mar. 3, 2005

(54) Imidazopyridines and triazolopyridines

(75) Inventors: Michael David Kaufman, Ypsilanti, MI (US); Mark Stephen Plummer, Dexter, MI (US); Gordon William Rewcastle, Glendowie (NZ)

Correspondence Address: AGOURON PHARMACEUTICALS, INC., 10350 NORTH TORREY PINES ROAD, LA JOLLA, CA, 92037, US

(73) Assignee: WARNER-LAMBERT COMPANY, LLC

(21) Appl. No.: 10/896,180

(22) Filed: Jul. 20, 2004

Related U.S. Application Data
(60) Provisional application No. 60/489,600, filed on Jul. 23, 2003.

(51) Int. Cl. C07D 471/04; A61K 31/4745

(52) U.S. Cl. 514/303; 546/118; 546/119

(57) ABSTRACT

The present invention relates to imidazopyridine and triazolopyridine derivatives, pharmaceutical compositions and methods of use thereof.

### FIELD OF THE INVENTION

This application claims the benefit of U.S. Provisional Application No. 60/489,600, filed Jul. 23, 2003.

The present invention relates to imidazopyridine and triazolopyridine derivatives, pharmaceutical compositions and methods of use thereof.

### BACKGROUND OF THE INVENTION

MAPK/ERK Kinase (“MEK”) enzymes are dual specificity kinases involved in, for example, immunomodulation, inflammation, and proliferative diseases such as cancer and restenosis.

Proliferative diseases are caused by a defect in the intracellular signaling system, or the signal transduction mechanism of certain proteins. Defects include a change either in the intrinsic activity or in the cellular concentration of one or more signaling proteins in the signaling cascade. The cell may produce a growth factor that binds to its own receptors, resulting in an autocrine loop, which continually stimulates proliferation. Mutations or overexpression of intracellular signaling proteins can lead to spurious mitogenic signals within the cell. Some of the most common mutations occur in genes encoding the protein known as Ras, a G-protein that is activated when bound to GTP, and inactivated when bound to GDP. The above-mentioned growth factor receptors, and many other mitogenic receptors, when activated, lead to Ras being converted from the GDP-bound state to the GTP-bound state. This signal is an absolute prerequisite for proliferation in most cell types. Defects in this signaling system, especially in the deactivation of the Ras-GTP complex, are common in cancers, and lead to the signaling cascade below Ras being chronically activated.

Activated Ras leads in turn to the activation of a cascade of serine/threonine kinases. One of the groups of kinases known to require an active Ras-GTP for its own activation is the Raf family. These in turn activate MEK (e.g., MEK₁ and MEK₂) which then activates the MAP kinase, ERK (ERK₁ and ERK₂). Activation of MAP kinase by mitogens appears to be essential for proliferation; constitutive activation of this kinase is sufficient to induce cellular transformation. Blockade of downstream Ras signaling, for example by use of a dominant negative Raf-1 protein, can completely inhibit mitogenesis, whether induced from cell surface receptors or from oncogenic Ras mutants. Although Ras is not itself a protein kinase, it participates in the activation of Raf and other kinases, most likely through a phosphorylation mechanism. Once activated, Raf and other kinases phosphorylate MEK on two closely adjacent serine residues, S²¹⁸ and S²²² in the case of MEK-1, which are the prerequisite for activation of MEK as a kinase. MEK in turn phosphorylates MAP kinase on both a tyrosine, y¹⁸⁵, and a threonine residue, T¹⁸³, separated by a single amino acid. This double phosphorylation activates MAP kinase at least 100-fold. Activated MAP kinase can then catalyze the phosphorylation of a large number of proteins, including several transcription factors and other kinaes. Many of these MAP kinase phosphorylations are mitogenically activating for the target protein, such as a kinase, a transcription factor, or another cellular protein. In addition to Raf-1 and MEKK, other kinases activate MEK, and MEK itself appears to be a signal integrating kinase. Current understanding is that MEK is highly specific for the phosphorylation of MAP kinase. In fact, no substrate for MEK other than the MAP kinase, ERK, has been demonstrated to date and MEK does not phosphorylate peptides based on the MAP kinase phosphorylation sequence, or even phosphorylate denatured MAP kinase. MEK also appears to associate strongly with MAP kinase prior to phosphorylating it, suggesting that phosphorylation of MAP kinase by MEK may require a prior strong interaction between the two proteins. Both this requirement and the unusual specificity of MEK are suggestive that it may have enough difference in its mechanism of action to other protein kinases that selective inhibitors of MEK, possibly operating through allosteric mechanisms rather than through the usual blockade of the ATP binding site, may be found.

It has been found that the compounds of the present invention are inhibitors of MEK and are useful in the treatment of a variety of proliferative disease states, such as conditions related to the hyperactivity of MEK, as well as diseases modulated by the MEK cascade.

### SUMMARY OF THE INVENTION

The present invention provides a compound of formula 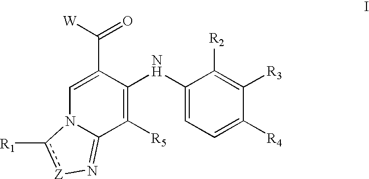
wherein
W is —OH, —O—(CH₂)ₖCH₃, —NH₂, —NH[(CH₂)ₖCH₃], or —NH[O(CH₂)ₖCH₃], wherein the —NH₂ is optionally substituted with between 1 and 2 substituents independently selected from methyl and amino, and the —(CH₂)ₖCH₃ moieties of the —O—(CH₂)ₖCH₃, —NH[(CH₂)ₖCH₃], and —NH[O(CH₂)ₖCH₃] groups are optionally substituted with between 1 and 3 substituents independently selected from hydroxy, amino, alkyl, and cycloalkyl;
Z is N, CH or CH₂:
the dashed line is an optional double bond, wherein the dashed line is a covalent double bond when Z is N or CH, and the dashed line is a covalent single bond when Z is CH₂;
R₁ is hydrogen, C₁₋₆ alkyl, C₂₋₄ alkenyl, C₂₋₆ alkynyl, aryl, heteroaryl, or —(CH₂)ₖO(CH₂)ₖOCH₃, wherein the C₁₋₆ alkyl is optionally substituted with between 1 and 2 substituents independently selected from hydroxy, —COOH, and cyano;
R₂ is hydrogen, chlorine, fluorine or methyl;
R₃ is hydrogen, chlorine, fluorine, methyl, or CF₃;
R₄ is bromine, chlorine, fluorine, iodine, C₁₋₆ alkyl, C₂₋₄ alkenyl, C₂₋₆ alkynyl, C₃₋₆ cycloalkyl, —(CH₂)—C₃₋₆ cycloalkyl, cyano, —O—(C₁₋₄alkyl), —S—(C₁₋₂ alkyl), —SOCH₃, —SO₂CH₃, —SO₂NR₆R₇, —C≡C—(CH₂)ₙNH₂, —C≡C—(CH₂)ₙNHCH₃, —C═C—(CH₂)ₙN(CH₃)₂, —C≡C—CH₂OCH₃, —C═C(CH₂)ₙOH, —C═C—(CH₂)ₙN H₂, —CHCHCH₂OCH₃, —CHCH—(CH₂)ₙNHCH₃, —CHCH—(CH₂)ₙN(CH₃)₂, —(CH₂)ₚCO₂R₆, C(O)C₁₋₃ alkyl, C(O)NHCH₃, —(CH₂)ₘNH₂, —(CH₂)ₘNHCH₃, —(CH₂)ₘN(CH₃)₂, —(CH₂)ₘOR₆, —CH₂S(CH₂)ₜ(CH₃), —(CH₂)ₚCF₃, —C≡CCF₃, —CH═CHCF₃, —CH₂CHCF₂, —CH═CF₂, —(CF₂)VCF₃, —CH₂(CF₂)ₙCF₃, —(CH₂)ₜCF(CF₃)₂, —CH(CF₃)₂, —CF₂CF(CF₃)₂, or —C(CF₃)₃, wherein the C₁₋₆ alkyl and C₂₋₆ alkynyl are optionally substituted with between 1 and 3 substituents independently selected from hydroxy and alkyl; or R₃ and R₄ can be joined together to form a six-membered aryl ring, five-membered cycloalkyl ring or a five or six-membered heteroaryl ring;
R₅ is hydrogen or fluorine;
R₆ and R₇ are each independently hydrogen, methyl, or ethyl;
k is 0 to 3;
m is 1 to 4;
n is 1 to 2;
p is 0 to 2;
t is 0 to 1;
v is 1 to 5;
and pharmaceutically acceptable salts, C₁₆ amides and C₁₆ esters thereof.

An embodiment of the present invention provide a compound of formula 1, as defined above, and pharmaceutically acceptable salts thereof.

Additionally provided by the present invention are compounds of Formula I, wherein W is —OH, —OCH₂CH₃, —NH₂, —NH [(CH₂)₂OH] or —NH[O(CH₂)₂OH]; or —OH, —OCH₂CH₃, —NH₂, or —NH [(CH₂)₂OH].

The present invention also provides compounds of Formula I wherein R₁ is hydrogen or methyl; R₁ is hydrogen; R₁ is methyl; R₂ is fluorine; R₃ is hydrogen; R₄ is iodine, C₁₃ alkyl, C₂₋₄ alkenyl, C₂₋₃ alkynyl, or —S—CH₃; R₄ is iodine; R₄ is ethyl, allyl or —S—CH₃; or R₅ is hydrogen.

Also provided by the present invention are compounds having a structure which is 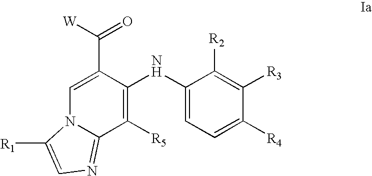
wherein W, R₁, R₂, R₃, R₄ and R₅ are defined as above.

Further provided by the present invention are compounds having a structure which is 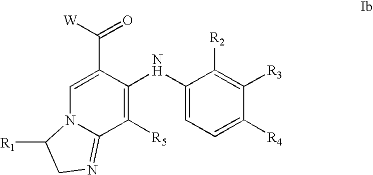
wherein W, R₁, R₂. R₃. R₄, and R₅ are defined as above.

Additionally provided by the present invention are compounds having a structure which is 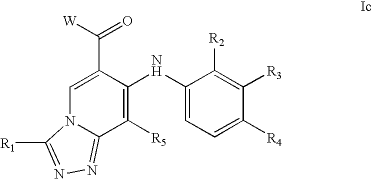
wherein W, R₁, R₂, R₃, R₄, and R₅ are defined as above.

The invention also provides a pharmaceutical composition comprising a compound of Formula I and a pharmaceutically acceptable carrier.

Additionally, the invention provides a method of treating a proliferative disease in a patient in need thereof comprising administering a therapeutically effective amount of a compound of Formula I.

Furthermore, the invention provides methods of treating cancer, restenosis, psoriasis, autoimmune disease, atherosclerosis, osteoarthritis, rheumatoid arthritis, heart failure, chronic pain, and neuropathic pain in a patient in need thereof comprising administering a therapeutically effective amount of a compound of Formula I.

In addition, the invention provides a method for treating cancer in a patient in need thereof comprising administering a therapeutically effective amount of a compound of Formula I in combination with radiation therapy or at least one chemotherapeutic agent.

The invention also provides the use of a compound of Formula I for the manufacture of a medicament for the treatment of the disease states or diseases provided above.

### DETAILED DESCRIPTION OF THE INVENTION

Certain terms are defined below and by their usage throughout this disclosure.

The terms “halogen” or “halo” in the present invention refer to a fluorine, bromine, chlorine, and iodine atom or fluoro, bromo, chloro, and iodo. The terms fluorine and fluoro, for example, are understood to be equivalent herein.

Alkyl groups, such as “C₁₋₆ alkyl”, include aliphatic chains (i.e., hydrocarbyl or hydrocarbon radical structures containing hydrogen and carbon atoms) with a free valence. Alkyl groups are understood to include straight chain and branched structures. Examples include methyl, ethyl, propyl, isopropyl, butyl, isobutyl, sec-butyl, t-butyl, pentyl, 2-pentyl, 3-pentyl, isopentyl, neopentyl, (R)-2-methylbutyl, (S)-2-methylbutyl, 3-methylbutyl, 2,3-dimethylpropyl, hexyl, and the like. The term “C₁₋₆ alkyl” includes within its definition the terms “C₁₋₄ alkyl” and “C₁₋₂ alkyl”.

The term “alkoxy” as used herein refers to a straight or branched alkyl chain attached to an oxygen atom. The term “C₁₋₈ alkoxy” as used herein refers to a straight or branched alkyl chain having from one to eight carbon atoms attached to an oxygen atom. Typical C₁₋₈ alkoxy groups include methoxy, ethoxy, propoxy, isopropoxy, butoxy, t-butoxy, pentoxy and the like. The term “C₁₋₈ alkoxy” includes within its definition the terms “C₁₋₆ alkoxy” and “C₁₋₄ alkoxy”.

Alkenyl groups are analogous to alkyl groups, but have at least one double bond (two adjacent Sp2 carbon atoms). Depending on the placement of a double bond and substituents, if any, the geometry of the double bond may be entgegen (E), or zusammen (Z), cis, or trans. Similarly, alkynyl groups have at least one triple bond (two adjacent sp carbon atoms). Unsaturated alkenyl or alkynyl groups may have one or more double or triple bonds, respectively, or a mixture thereof. Like alkyl groups, unsaturated groups may be straight chain or branched. Examples of alkenyls and alkynyls include vinyl, allyl, 2-methyl-2-propenyl, cis-2-butenyl, trans-2-butenyl, and acetyl.

Cycloalkyl groups, such as C₃₋₆ cycloalkyl, refer to a saturated hydrocarbon ring structure containing from 3 to 6 atoms. Typical C₃₋₆ cycloalkyl groups include cyclopropyl, cyclobutyl, cyclopentyl, cyclohexyl, and the like.

The term “aryl” means an unsubstituted aromatic carbocyclic group having a single ring (e.g., phenyl), multiple rings (e.g., biphenyl), or multiple condensed rings in which at least one is aromatic (e.g., 1,2,3,4-tetrahydronaphthyl, naphthyl, anthryl, or phenanthryl).

The term “heteroaryl”, as used herein, unless otherwise indicated, includes monocyclic aromatic heterocycles containing five or six ring members, of which from 1 to 4 can be heteroatoms selected, independently, from N, S and O, and bicyclic aromatic heterocycles containing from eight to twelve ring members, of which from 1 to 4 can be heteroatoms selected, independently, from N, S and O.

Heterocyclic radicals, which include but are not limited to heteroaryls, include: furyl, (is)oxazolyl, isoxazolyl, thiophenyl, thiazolyl, pyrrolyl, imidazolyl, 1,3,4-triazolyl, tetrazolyl, pyridinyl, pyrimidinyl, pyridazinyl, indolyl, and their nonaromatic counterparts. Further examples of heterocyclic radicals include thienyl, piperidyl, quinolyl, isothiazolyl, piperidinyl, morpholinyl, piperazinyl, tetrahydrofuryl, tetrahydropyrrolyl, pyrrolidinyl, octahydroindolyl, octahydrobenzothiofuranyl, octahydrobenzofuranyl, (iso)quinolinyl, naphthyridinyl, benzimidazolyl, and benzoxazolyl.

The present invention includes the hydrates and the pharmaceutically acceptable salts and solvates of the compounds defined by Formula I. The compounds of this invention can possess a sufficiently basic functional group, and accordingly react with any of a number of inorganic and organic acids, to form a pharmaceutically acceptable salt.

The term “pharmaceutically acceptable salt” as used herein, refers to salts of the compounds of Formula I which are substantially non-toxic to living organisms. Typical pharmaceutically acceptable salts include those salts prepared by reaction of the compounds of the present invention with a pharmaceutically acceptable mineral or organic acid. Such salts are also known as acid addition salts. Such salts include the pharmaceutically acceptable salts listed in Journal of Pharmaceutical Science, 1977;66:2-19, which are known to the skilled artisan.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, and the like. Example of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, hydrobromide, iodide, acetate, propionate, decanoate, caprate, caprylate, acrylate, ascorbate, formate, hydrochloride, dihydrochloride, isobutyrate, caproate, heptanoate, propiolate, glucuronate, glutamate, propionate, phenylpropionate, salicylate, oxalate, malonate, succinate, suberate, sebacate, fumarate, malate, maleate, hydroxymateate, mandelate, mesylate, nicotinate, isonicotinate, cinnamate, hippurate, nitrate, stearate, phthalate, teraphthalate, butyne-1,4-dioate, butyne-1,4-dicarboxylate, hexyne-1,4-dicarboxylate, hexyne-1,6-dioate, benzoate, chlorobenzoate, methylbenzoate, hydrozybenzoate, methoxybenzoate, dinitrobenzoate, o-acetoxybenzoate, naphthalene-2-benzoate, phthalate, p-toluenesulfonate, p-bromobenzenesulfonate, p-chlorobenzenesulfonate, xylenesulfonate, phenylacetate, trifluoroacetate, phenylpropionate, phenylbutyrate, citrate, lactate, a-hydroxybutyrate, glycolate, tartrate, hemi-tartrate, benzenesulfonate, methanesulfonate, ethanesulfonate, propanesulfonate, hydroxyethanesulfonate, 1-naphthalenesulfonate, 2-naphthalenesulfonate, 1,5-naphthalenedisulfonate, mandelate, tartarate, and the like. A preferred pharmaceutically acceptable salt is hydrochloride.

It should be recognized that the particular counterion forming a part of any salt of this inventions is usually not of a critical nature, so long as the salt as a whole is pharmacologically acceptable and as long as the counterion does not contribute undesired qualities to the salt as a whole. It is further understood that such salts may exist as a hydrate.

The enantiomers of compounds of the present invention can be resolved by one of ordinary skill in the art using standard techniques well-known in the art, such as those described by J. Jacques, et al., “Enantiomers, Racemates, and Resolutions”, John Wiley and Sons, Inc 1981. Examples of resolutions include recrystallization techniques or chiral chromatography.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The compounds of Formula I can be prepared by techniques and procedures readily available to one of ordinary skill in the art, for example by following the procedures as set forth in the following Schemes, or analogous variants thereof. These synthetic strategies are further exemplified in examples below. These schemes are not intended to limit the scope of the invention in any way.

As used herein, the following terms have the meanings indicated: “AcOH” refers to acetic acid; “CDI” refers to 1,1′-carbonyldiimidazole; Celite® refers to a filter agent which is acid washed and approximately 95% SiO₂; “CHCl₃” refers to chloroform; “CH₂Cl₂” and “DCM” refer to dichloromethane; “conc.” refers to concentrated; “DABCO” refers to 1,4-diazabicyclo[2.2.2]octane; “DIEA” refers to N,N-diisopropylethylamine; “DMA” refers to N,N-dimethylacetamide; “DMF” refers to N,N-dimethylformamide; “DMSO” refers to methyl sulfoxide; “DMT-M M” refers to 4-(4,6-dimethoxy-1,3,5-triazin-2-yl)-4-methylmorpholinium chloride; “EtOAc” refers to ethyl acetate; “EtOH” refers to ethanol; “Et₂O” refers to diethyl ether; “FMOC” refers to 9H-fluoren-9-ylmethyl ester; “h” refers to hours; “HCl” refers to hydrochloric acid; “Me” refers to methyl; “MeOH” refers to methanol; “Me₂SO₄” refers to dimethyl sulfate; “min” refers to minutes; “NaOH” refers to sodium hydroxide “Na₂SO₄ refers to sodium sulfate; “N-MM” refers to N-methylmorpholine; “Pd/C” refers to palladium on carbon; “PE” refers to petroleum ether which can be substituted with hexanes; “(Ph₃P)₂PdCl₂” refers to dichlorobis (triphenylphosphine)palladium(II); “(Ph₃P)₄Pd” refers to tetrakis (triphenylphosphine) palladium (0); “PS” refers to polymer—supported; “R.T.” refers to room temperature; “sat” refers to saturated; “TEA” refers to triethylamine; “TFA” refers to trifluoroacetic acid; “THF” refers to “tetrahydrofuran; “TLC” refers to thin layer chromatography and “TMS” refers to trimethylsilyl. All other terms and substituents, unless otherwise indicated, are previously defined.

All other terms and substituents, unless otherwise indicated, are previously defined. The reagents and starting materials are readily available to one of ordinary skill in the art. Schemes 1 and 2a-c provide syntheses of the compounds of Formula I.

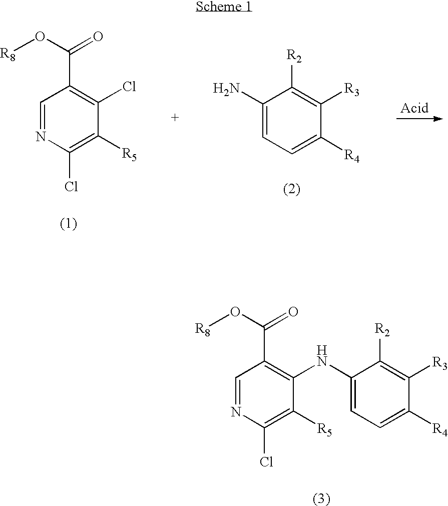

### R₈ is hydrogen, alkyl or substituted alkyl

In Scheme 1, a suitable dichloronicotinic ester (1) is coupled with a suitable aniline (2) to provide a 4-(arylamino)nicotinate (3). For example, the aniline (2) and the dichloronicotinate (1) are dissolved in a suitable organic solvent with an acid catalyst and heated at reflux for several hours. Preferred solvents are polar solvents such as ethanol, and preferred acid catalysts are mineral acids such as concentrated HCl. The reaction is typically complete within about 12 to 36 hours. The product ester (3) is typically isolated by filtration after cooling of the reaction mixture, and further purified, if desired, by standard methods such as chromatography or crystallization. 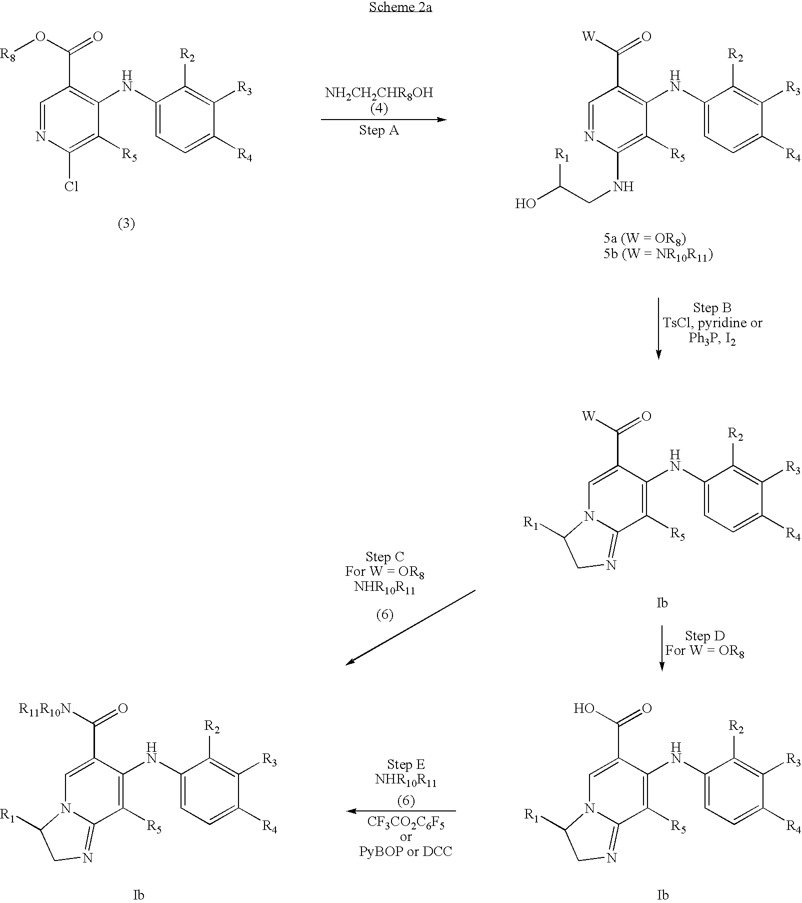
R₈ is hydrogen, alkyl or substituted alkyl
R₁₀ and R₁₁ are independently hydrogen, amino, alkyl, substituted alkyl, alkoxy or substituted alkoxy

Scheme 2a shows the conversion of compound (3) to a compound of formula Ib. In step A, contact of chloropyridine (3) with a suitable ethanolamine (4) affords product aminopyridines (5). Choice of reaction solvent, temperature, and length of reaction determine whether W remains OR₈ (5a) or is converted to NR₁₀R₁₁ (5b). Preferred solvents include ethanol and toluene at temperatures between 80° C. and 120° C. In step B, the dihydroimidazole ring is constructed by intramolecular N-alkylation upon in situ activation of the alcohol (5), providing compounds of formula Ib. Preferred methods of activation include conversion the alcohol to a tosylate, methanesulfonate, trifluormethansulfonate, or iodide, using reagents known to those skilled in the art. Especially preferred reagents include p-toluenesulfonyl chloride in pyridine or triphenylphosphine-iodine in tetrahydrofuran. Finally, the nature of the W group may be changed by a variety of conditions to those skilled in the art. For example, an ester of formula Ib may be converted to a carboxylic acid of formula Ib by the action of a suitable hydroxide (step D), such as potassium hydroxide. Alternately, an ester of formula Ib may be converted to an amide of formula Ib by aminolysis (step C), for example using ammonia in methanol at elevated temperature. Amides of formula Ib can also be generated by union of an acid of formula Ib, where W is hydroxy, and amine (6) mediated by standard coupling agents such as PyBOP or dicyclohexylcarbodiimide (DCC) or via the intermediacy of mixed anhydrides or activated esters, for example pentafluorophenyl esters (step E).

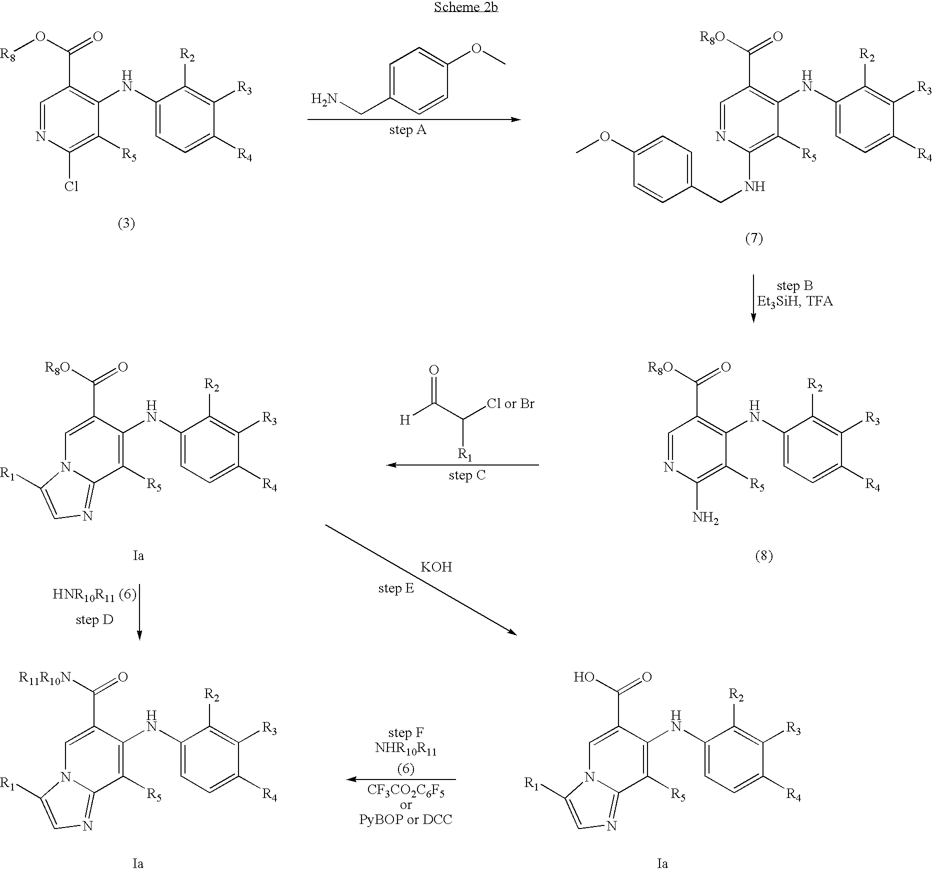

Scheme 2b illustrates the conversion of intermediate (3) into compounds of formula Ia. In step A, chloropyridine (3) is converted to p-methoxybenzylamine (7) by reaction with p-methoxybenzylamine. Removal of the p-methoxybenzyl protecting group affords aminopyridines (8) (step B). In step C, the treatment of aminopyridine (8) with a-chloro or bromo-substituted aldehydes affords imidazopyridines of formula Ia, wherein W is —OR₈. Other compounds of the invention are accessible from formula Ia, wherein W is —OR₈, by aminolysis (step D) or saponification (step E) as indicated in Scheme 3. Compounds of formula Ia, wherein W is NR₁₀R₁₁, may also be prepared by union of acid of formula Ia with amine (6) mediated by standard coupling agents such as PyBOP or dicyclohexylcarbodiimide (DCC) or via the intermediacy of mixed anhydrides or activated esters, for example pentafluorophenyl esters (step F).

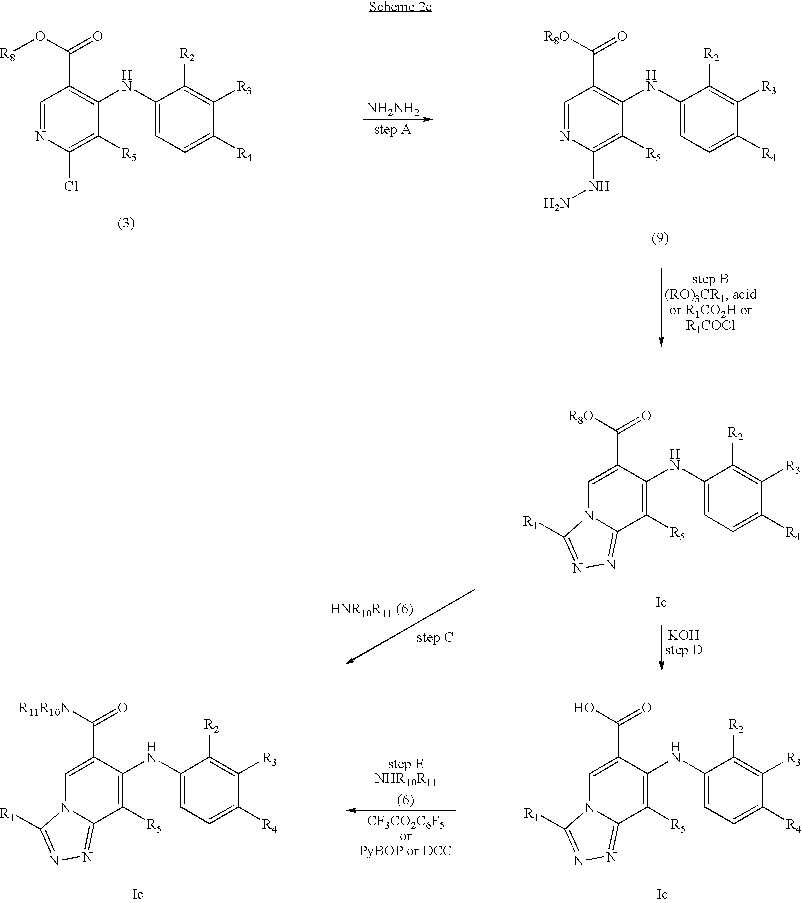

Scheme 2c illustrates the conversion of intermediate (3) to triazolopyridines of formula Ic. In step A, hydrazine is added to chloropyridine (3). In step B, the triazole ring is formed by reaction of hydrazide (9) with a carboxylic acid or acid equivalent, such as an orthoester, acid halide or acid anhydride to afford compounds of formula Ic, wherein W is —OR₈. Other compounds of the invention are accessible from formula Ic, wherein W is —OR₈ by aminolysis (step C) or saponification (step D) as indicated in scheme 4. Compounds of formula Ia, wherein W is NR₁₀R₁₁, may also be prepared by union of acid of formula Ia with amine (6) mediated by standard coupling agents such as PyBOP or dicyclohexylcarbodiimide (DCC) or via the intermediacy of mixed anhydrides or activated esters, for example pentafluorophenyl esters (step F).

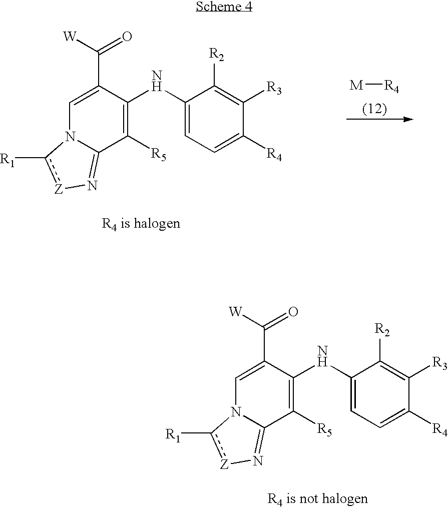

In Scheme 4, the compounds of formula 1, wherein R₄ is not halogen are prepared from the compounds of formula I wherein R₄ is halogen, by transition metal-promoted coupling with reagent M-R₄ wherein R₄ is non-halogen (12) in a suitable solvent or solvents such as triethylamine, tetrahydrofuran or dimethylformamide. The transition metal-promoted coupling may be carried out with a palladium(0) or palladium (II) coupling agent, such as (Ph₃P)₄Pd or (Ph₃P)₂PdCl₂. The entire mixture is stirred from about 2 to 24 hours at room temperature. M is defined as a functional group known to transfer a carbon radical fragment in transition metal-promoted coupling processes. Examples of a suitable M group include trialkylstannyl, trialkylsilyl, trimethylsilyl, zinc, tin, copper, boron, magnesium and lithium. Examples of a suitable M-R₄ reagent (12) when, R₄ is C₂₋₄ alkenyl is allyltributyltin or tetravinyltin, and when R₄ is hydroxy-substituted C₂₋₆ alkynyl is propargyl alcohol. Preferred halogens, when R₄ is halogen, are bromine and iodine.

The resulting compound of formula 1, as well as the protected Formula I compound, can be isolated by removing the solvent, for example by evaporation under reduced pressure, and further purified, if desired, by standard methods such as chromatography, crystallization, or distillation.

It would be understood by one of skill in the art that the substituent R₄, when R₄ is non-halogen, may be further transformed, such as by oxidation, reduction, deprotection, or hydrogenation.

A compound wherein R₄ is C₂₋₄ alkenyl may be transformed to a compound wherein R₄ is hydroxy-substituted alkyl by treating the double bond of the alkene with ozone and NaBH₄. Furthermore, a compound wherein R₄ is C₂₋₄ alkenyl may be transformed to a compound wherein R₄ is alkyl substituted with 2 hydroxy substituents by treating the double bond of the alkene with OsO₄.

A compound wherein R₄ is an alkene or alkyne derivative may be reduced under conditions known in the art, such as through hydrogenation, such as with Pd/C under an atmosphere of hydrogen. For example, the alkyne derivative is dissolved in a suitable solvent, such as absolute ethanol, in the presence of a metal catalyst, such as palladium on carbon. This mixture is stirred under an atmosphere of hydrogen from about 1 to 24 hours at room temperature to provide the fully saturated derivative. Alternately, the alkyne derivative is partially reduced via hydrogenation to provide the alkene derivative. For example, the alkyne derivative is dissolved in a suitable solvent, such as tetrahydrofuran, in the presence of a catalyst, such as Lindlar catalyst or palladium on carbon and, if desired, a suitable compound which disrupts the actions of the catalyst, such as quinoline or pyridine. This mixture is stirred under an atmosphere of hydrogen from about 1 to 24 hours at room temperature to provide the alkene derivative.

The substituent R₄ may also be transformed into a different R₄ through standard synthetic procedures known to one of skill in the art.

It would be understood by one of skill in the art that the transformation of R₄ as shown in Scheme 4 may be performed at various steps throughout the synthesis of compounds of the present invention, as desired. For example, R₄ may be transformed before the coupling of the ester (1) and aniline (2) as shown in Scheme 1, Step A.

Further transformations of R₄ are shown in Scheme 5 below.

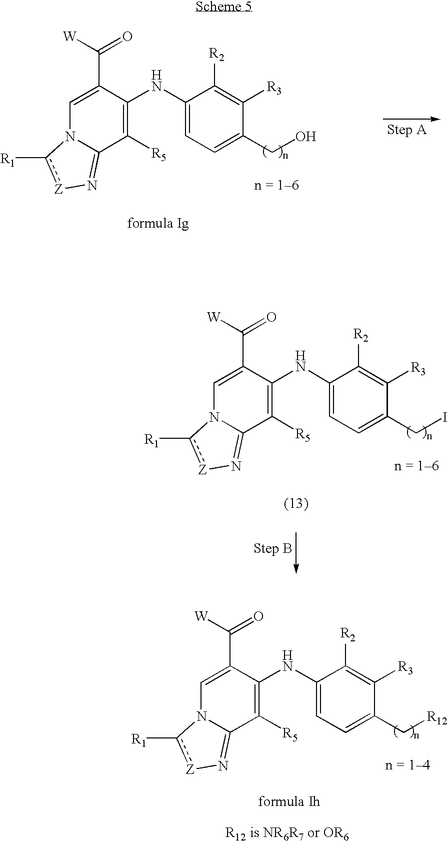

In Scheme 5, step A, the compound of formula Ig is dissolved in a suitable solvent such as tetrahydrofuran and reacted with methanesulfonyl chloride to give the intermediate mesylate, then NaI in EtOAc to give the iodide compound (13).

In Scheme 5, step B, the iodide compound (13) is reacted with a suitable amine, such as methylamine or dimethylamine, or a suitable alkoxide to give compounds of formula Ih.

It would also be understood by one of skill in the art that an aniline (2) may be prepared to include the desired R₄.

The aniline (2) can be prepared by techniques and procedures readily available to one of ordinary skill in the art and by following the procedures as set forth in the following Schemes, or analogous variants thereof. Additionally, anilines (2) are taught in U.S. Ser. No. 10/349,801 filed Jan. 23, 2003 and U.S. Ser. No. 10/349,826 filed Jan. 23, 2003, the disclosure of which is hereby incorporated by reference. These Schemes are not intended to limit the scope of the invention in any way.

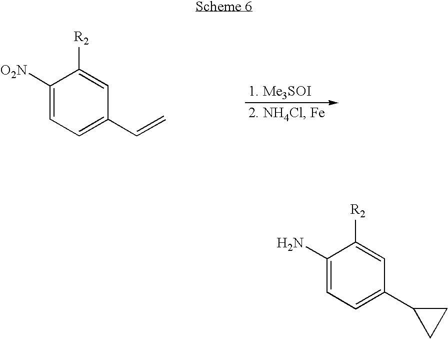

### Bull. Soc. Chim. Belg., 95(2), 135-8; 1986

In Scheme 6, a suitably substituted para-nitrostyrene is reacted with dimethyloxosulfonium methylide to form the substituted para-nitrocyclopropylbenzene. Reduction of para-nitrocyclopropylbenzene with iron in the presence of weak acid gives the desired aniline.

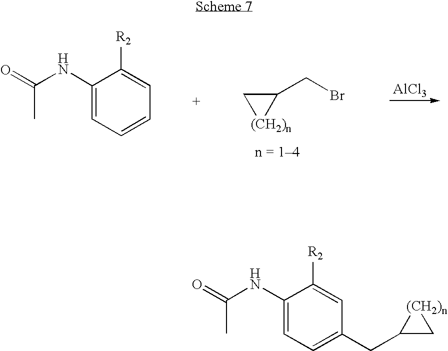

In Scheme 7, the suitable ortho-substituted acetamide is reacted with bromocyclobutane, bromocyclopropane, or bromocyclohexane under typical Friedel-Craft conditions, as known to one of skill in the art, to give the desired para-cycloalkylanilines. The acetamide is deprotected under conditions known to one of skill in the art to provide the desired para-cycloalkylmethylanilines.

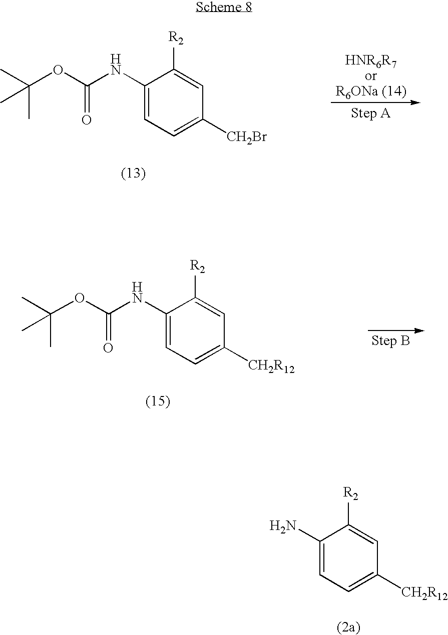

In Scheme 8, Step A, a suitable amine or alkoxide (14) is reacted with a 4-tert-butoxycarbonylamino-3-substituted-benzyl bromide (13), such as 4-tert-butoxycarbonylamino-3-fluorobenzyl bromide (J. Med. Chem., 2000;43:5017). In Step B, the BOC protecting group of compound of structure (15) is hydrolized with, for example, TFA, to provide the desired aniline (2a).

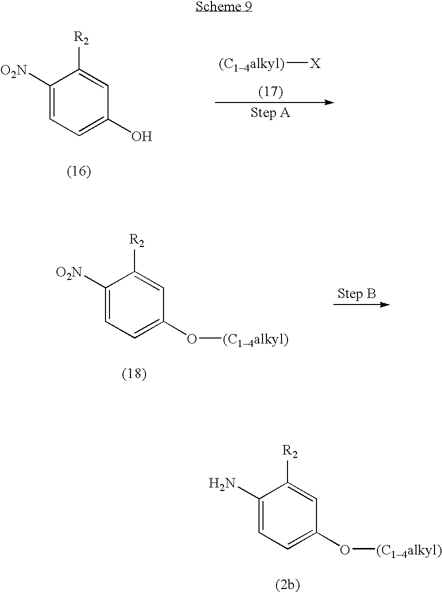

In Scheme 9, Step A, a suitable 3-substituted-4-nitrophenol (16), such as 3-fluoro-4-nitrophenol, is alkylated with a compound of structure (17) in the presence of a suitable base to provide a compound of structure (18). In Step B, compound (18) is reduced via hydrogenation in the presence of a metal catalyst, such as palladium on carbon, in an atmosphere of hydrogen to provide the desired aniline (2b).

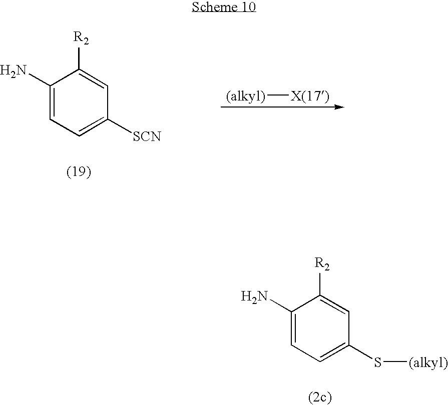

In Scheme 10, a suitable 4-(aminophenyl)thiocyanate (19), is alkylated with a compound of structure (17′) in the presence of a suitable nucleophilic base to provide an alkylthio compound of structure (2c). After reaction under standard conditions to form the diphenylamine (3), wherein R₄ is —S-(alkyl), as in Scheme 1 above, this compound is then oxidized to the corresponding sulfonyl compound, also generally, the diphenylamine (3), wherein R₄ is —SO₂— (alkyl).

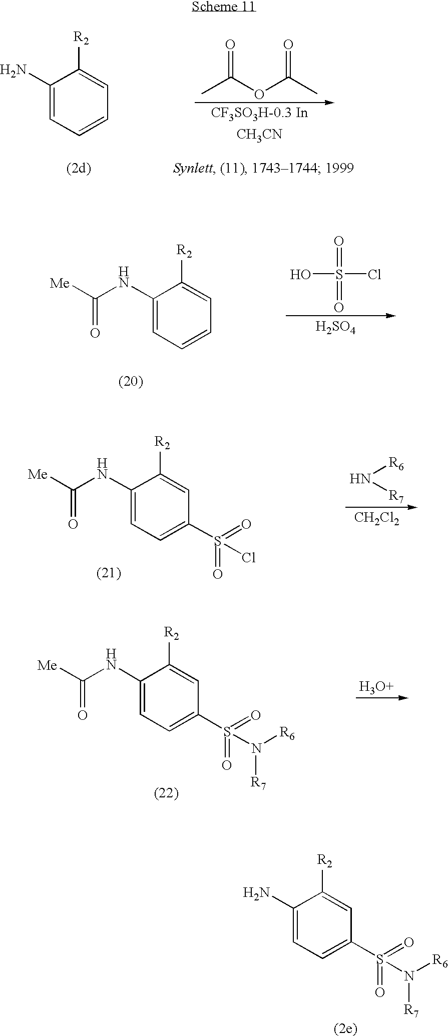

In Scheme 11, the proper ortho-substituted or unsubstituted aniline (2d) is acetylated with acetic anhydride in the presence of trifluoromethanesulfonic acid indium salt to give the protected aniline (20). Chlorosulfonation in the typical manner, as known in the art, gives the sulfonyl chloride derivative (21) which is reacted with an excess of a suitable amine in a solvent such as dichloromethane or dichloroethane to give the protected para-aminobenzenesulfonamide (22). Acid-mediated deprotection in the appropriate solvent gives the desired aniline (2e).

Alternatively, the desired aniline (2e) wherein R₂ is methyl, fluorine or chlorine, using compound (21) as the starting material can be prepared. Where R₂ is fluorine, the sulfonyl chloride derivative (21) is a compound known in the literature (German Patent DE 2630060, 1978). Similarly, where R₂ is methyl, the sulfonyl chloride derivative (21) is also known in the literature (German Patent. DE 2750170, 1978). Finally, the sulfonyl chloride derivative (21) where R₂ is chlorine is commercially available.

In addition to the procedure described in Scheme 11, one of ordinary skill in the art would appreciate that there are numerous ways of acetylating anilines. For example, heating the aniline and acetic anhydride together in a suitable solvent, such as acetic acid, would achieve the same result.

Compounds of the present invention include, but are not limited to the following compounds:
7-(2-Fluoro-4-iodo-phenylamino)-imidazo[1,2-a]pyridine-6-carboxylic acid ethyl ester;
7-(2-Fluoro-4-iodo-phenylamino)-imidazo[1,2-a]pyridine-6-carboxylic acid;
7-(2-Fluoro-4-iodo-phenylamino)-imidazo[1,2-a]pyridine-6-carboxylic acid amide;
7-(2-Fluoro-4-iodo-phenylamino)-2,3-dihydro-imidazo[1,2-a]pyridine-6-carboxylic acid (2-hydroxy-ethyl)-amide;
7-(2-Fluoro-4-iodo-phenylamino)-3-methyl-[1,2,4]triazolo[4,3-a]pyridine-6-carboxylic acid ethyl ester;
7-(2-Fluoro-4-iodo-phenylamino)-3-methyl-[1,2,4]triazolo[4,3-a]pyridine-6-carboxylic acid amide; and
7-(2-Fluoro-4-iodo-phenylamino)-2,3-dihydro-imidazo[1,2-a]pyridine-6-carboxylic acid ethyl ester.

As used herein, the term “patient” refers to any warm-blooded animal such as, but not limited to, a human, horse, dog, guinea pig, or mouse. Preferably, the patient is human.

The term “treating” for purposes of the present invention refers to treatment, prophylaxis or prevention, amelioration or elimination of a named condition once the condition has been established.

Selective MEK 1 or MEK 2 inhibitors are those compounds which inhibit the MEK 1 or MEK 2 enzymes, respectively, without substantially inhibiting other enzymes such as MKK3, PKC, Cdk2A, phosphorylase kinase, EGF, and PDGF receptor kinases, and C-src. In general, a selective MEK 1 or MEK 2 inhibitor has an IC₅₀ for MEK 1 or MEK 2 that is at least one-fiftieth (1/50) that of its IC₅₀ for one of the above-named other enzymes. Preferably, a selective inhibitor has an IC₅₀ that is at least 1/100, more preferably 1/500, and even more preferably 1/1000, 1/5000, or less than that of its IC₅₀ or one or more of the above-named enzymes.

The disclosed compositions are useful as both prophylactic and therapeutic treatments for diseases or conditions related to the hyperactivity of MEK, as well as diseases or conditions modulated by the MEK cascade. Examples include, but are not limited to, stroke, septic shock, heart failure, osteoarthritis, rheumatoid arthritis, organ transplant rejection, and a variety of tumors such as ovarian, lung, pancreatic, brain, prostatic, and colorectal.

The invention further relates to a method for treating proliferative diseases, such as cancer, restenosis, psoriasis, autoimmune disease, and atherosclerosis. Other aspects of the invention include methods for treating MEK-related (including ras-related) cancers, whether solid or hematopoietic. Examples of cancers include brain, breast, lung, such as non-small cell lung, ovarian, pancreatic, prostate, renal, colorectal, cervical, acute leukemia, and gastric cancer. Further aspects of the invention include methods for treating or reducing the symptoms of xenograft (cell(s), skin, limb, organ or bone marrow transplant) rejection, osteoarthritis, rheumatoid arthritis, cystic fibrosis, complications of diabetes (including diabetic retinopathy and diabetic nephropathy), hepatomegaly, cardiomegaly, stroke (such as acute focal ischemic stroke and global cerebral ischemia), heart failure, septic shock, asthma, Alzheimer's disease, and chronic or neuropathic pain. Compounds of the invention are also useful as antiviral agents for treating viral infections such as HIV, hepatitis (B) virus (HBV), human papilloma virus (HPV), cytomegalovirus (CMV), and Epstein-Barr virus (EBV). These methods include the step of administering to a patient in need of such treatment, or suffering from such a disease or condition, a therapeutically effective amount of a disclosed compound of formula I or pharmaceutical composition thereof.

The term “chronic pain” for purposes of the present invention includes, but is not limited to, neuropathic pain, idiopathic pain, and pain associated with chronic alcoholism, vitamin deficiency, uremia, or hypothyroidism. Chronic pain is associated with numerous conditions including, but not limited to, inflammation, arthritis, and post-operative pain.

As used herein, the term “neuropathic pain” is associated with numerous conditions which include, but are not limited to, inflammation, postoperative pain, phantom limb pain, burn pain, gout, trigeminal neuralgia, acute herpetic and postherpetic pain, causalgia, diabetic neuropathy, plexus avulsion, neuroma, vasculitis, viral infection, crush injury, constriction injury, tissue injury, limb amputation, arthritis pain, and nerve injury between the peripheral nervous system and the central nervous system.

The invention also features methods of combination therapy, such as a method for treating cancer, wherein the method further includes providing radiation therapy or chemotherapy, for example, with mitotic inhibitors such as a taxane or a vinca alkaloid. Examples of mitotic inhibitors include paclitaxel, docetaxel, vincristine, vinblastine, vinorelbine, and vinflunine. Other therapeutic combinations include a MEK inhibitor of the invention and an anticancer agent such as cisplatin, 5-fluorouracil or 5-fluoro-2-4(1H, 3H)-pyrimidinedione (5FU), flutamide, and gemcitabine.

The chemotherapy or radiation therapy may be administered before, concurrently, or after the administration of a disclosed compound according to the needs of the patient.

Those skilled in the art will be able to determine, according to known methods, the appropriate therapeutically-effective amount or dosage of a compound of the present invention to administer to a patient, taking into account factors such as age, weight, general health, the compound administered, the route of administration, the type of pain or condition requiring treatment, and the presence of other medications. In general, an effective amount or a therapeutically-effective amount will be between about 0.1 and about 1000 mg/kg per day, preferably between about 1 and about 300 mg/kg body weight, and daily dosages will be between about 10 and about 5000 mg for an adult subject of normal weight. Commercially available capsules or other formulations (such as liquids and film-coated tablets) of 100, 200, 300, or 400 mg can be administered according to the disclosed methods.

The compounds of the present invention are preferably formulated prior to administration. Therefore, another aspect of the present invention is a pharmaceutical composition comprising a compound of Formula I and a pharmaceutically acceptable carrier. In making the compositions of the present invention, the active ingredient, such as a compound of Formula I, will usually be mixed with a carrier, or diluted by a carrier or enclosed within a carrier. Dosage unit forms or pharmaceutical compositions include tablets, capsules, pills, powders, granules, aqueous and nonaqueous oral solutions and suspensions, and parenteral solutions packaged in containers adapted for subdivision into individual doses.

Dosage unit forms can be adapted for various methods of administration, including controlled release formulations, such as subcutaneous implants. Administration methods include oral, rectal, parenteral (intravenous, intramuscular, subcutaneous), intracisternal, intravaginal, intraperitoneal, intravesical, local (drops, powders, ointments, gels, or cream), and by inhalation (a buccal or nasal spray).

Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Carriers for solid dosage forms include (a) fillers or extenders, (b) binders, (c) humectants, (d) disintegrating agents, (e) solution retarders, (f) absorption acccelerators, (g) adsorbants, (h) lubricants, (i) buffering agents, and (j) propellants.

Compositions may also contain adjuvants such as preserving, wetting, emulsifying, and dispensing agents; antimicrobial agents such as parabens, chlorobutanol, phenol, and sorbic acid; isotonic agents such as a sugar or sodium chloride; absorption-prolonging agents such as aluminum monostearate and gelatin; and absorption-enhancing agents.

The following examples represent typical syntheses of the compounds of the present invention as described generally above. These examples are illustrative only and are not intended to limit the invention in any way. The reagents and starting materials are readily available to one of ordinary skill in the art.

### EXAMPLE 1

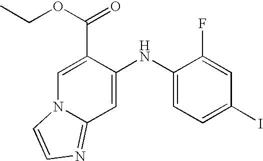 7-(2-Fluoro-4-iodo-phenylamino)-imidazo[1,2-a]pyridine-6-carboxylic acid ethyl ester 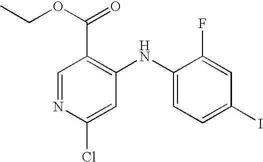
Step A: Preparation of ethyl 6-chloro-4-(2-fluoro-4-iodoanilino)nicotinate

Ethyl 4,6-dichloronicotinate [prepared according to the literature procedure of J. Chem. Soc. 5163 (1963)] (15.0 g, 68.1 mmol) and 2-fluoro-4-iodoaniline (15.0 g, 63.3 mmol) were dissolved in ethanol (100 mL), to which was added conc. hydrochloric acid (4 drops). This mixture was heated at reflux for 12 h. The solution was allowed to cool to ambient temperature. The resultant solid was slurried with ethanol (50 mL) and was filtered. The filter cake was further washed with ethanol (2×50 mL) and dried in vacuo to give ethyl 6-chloro-4-(2-fluoro-4-iodoanilino)nicotinate (15.88 g, 60% yield): m.p. (EtOAc/n-hexane) 162-164° C. ¹H NMR [(CD₃)₂SO, 400 MHz] δ 9.62 (s, 1H), 8.69 (s, 1H), 7.82 (dd, J=9.9, 1.9 Hz, 1H), 7.61-7.66 (m, 1H), 7.33 (t, J=8.5 Hz, 1H), 6.67 (d, J=1.8 Hz), 4.37 (q, J=7.1 Hz, 2H), 1.35 (t, J=7.1 Hz, 3H). Anal. Calcd for C₁₄H₁₁CIFIN₂O₂: C, 40.0; H, 2.6; N, 6.7. Found: C, 40.3; H, 2.2; N, 6.7. 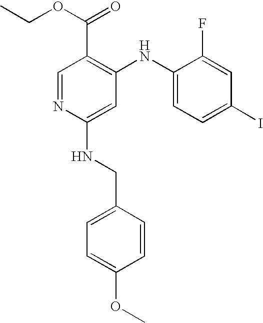
Step B: Preparation of 4-(2-Fluoro-4-iodo-phenylamino)-6-(4-methoxy-benzylamino)-nicotinic acid ethyl ester

A solution of 4-methoxybenzylamine (0.60 mL, 4.59 mol) and ethyl 6-chloro-4-(2-fluoro-4-iodoanilino)nicotinate (1.54 g, 3.65 mmol) in toluene (10 mL) was heated at reflux for 42 h. The reaction mixture was cooled to R. T., affording a white precipitate. The solvent was decanted and the solid was triturated with boiling toluene (10 mL) for 5 min. After cooling to ambient temperature, the solvent was removed affording 4-(2-Fluoro-4-iodo-phenylamino)-6-(4-methoxy-benzylamino)-nicotinic acid ethyl ester (0.554 g) as a white powder. The toluene mother liquors were combined, concentrated in vacuo and chromatographed on silica gel to afford an additional crop of 4-(2-Fluoro-4-iodo-phenylamino)-6-(4-methoxy-benzylamino)-nicotinic acid ethyl ester (0.364 g, 48% total yield). 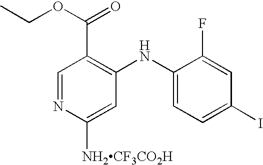
Step C: Preparation of the trifluoroacetate salt of 6-Amino-4-(2-fluoro-4-iodo-phenylamino)-nicotinic acid ethyl ester

Trifluoroacetic acid (10 mL) was added to a solution of 4-(2-Fluoro-4-iodo-phenylamino)-6-(4-methoxy-benzylamino)-nicotinic acid ethyl ester (0.913 g, 1.75 mmol) and triethylsilane (0.84 mL, 5.3 mmol) in dichloromethane (10 mL). The resultant orange-colored reaction mixture was stirred 4 h at ambient temperature. The solvent was removed in vacuo and the resultant oil was diluted with ether (20 mL). Upon standing, crystals of 4-methoxybenzylamine trifluoracetate formed. The crystals were removed by filtration and the filtrate was concentrated in vacuo to afford an oil that solidified upon standing. The solid was dissolved in warm ethyl acetate. Upon cooling, crystals formed. The crystals were collected, washed with ether and dried in vacuo, providing the trifluoracetate salt of 6-Amino-4-(2-fluoro-4-iodo-phenylamino)-nicotinic acid ethyl ester (0.394 g). The crystallization procedure was repeated affording a second crop (0.18, 64% total yield).

Step D: Preparation of 7-(2-Fluoro-4-iodo-phenylamino)-imidazo[1,2-a]pyridine-6-carboxylic acid ethyl ester

A solution comprised of the trifluoracetate salt of 6-Amino-4-(2-fluoro-4-iodo-phenylamino)-nicotinic acid ethyl ester (0.37 g, 0.72 mmol), ethanol (3.8 mL) and aqueous chloroacetaldehyde (50 wt %, 0.8 mL, 6.3 mmol) was heated to 70° C. After 25 min, potassium carbonate (47 mg, 0.34 mmol) was added and heating was continued. After 30 min, a second portion of potassium carbonate (55 mg, 0.40 mmol) was added and the reaction was heated at 70° C. for 1 h. The reaction mixture was cooled to ambient temperature and partitioned between ethyl acetate and water. The organics were washed with 10% aqueous sodium hydroxide and brine, dried over magnesium sulfate and concentrated in vacuo. Silica gel chromatography (10% methanol-dichloromethane→20% methanol-dichlorormethane, 30 min) afforded 7-(2-Fluoro-4-iodo-phenylamino)-imidazo[1,2-a]pyridine-6-carboxylic acid ethyl ester (0.17 g, 56% yield) as a yellow solid: m.p. 132-134° C. (ethyl acetate-ether); ¹H NMR (400 MHz, DMSO-d₆) 69.31(s, 1H), 9.07 (s, 1H), 7.88 (m, 1H), 7.73 (dd, J=10.5, 2.0 Hz, 1H), 7.56 (dm, J=8.5 Hz, 1H), 7.45 (d, J=1.5 Hz, 1H), 7.40 (t, J=8.5 Hz, 1H), 6.84 (br s, 1H), 4.36 (q, J=7.1 Hz, 2H), 1.34 (t, J=7.1 Hz, 3H); ¹⁹F NMR (376 MHz, DMSO-d₆) 6-124.5; MS (APCI+) 426.0 [M+1]. RMECASC IC₅₀=0.0239 μM

### EXAMPLE 2

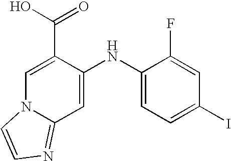 7-(2-Fluoro-4-iodo-phenylamino)-imidazo[1.2-a]pyridine-6-carboxylic acid

A solution of 7-(2-Fluoro-4-iodo-phenylamino)-imidazo[1,2-a]pyridine-6-carboxylic acid ethyl ester (160 mg, 0.38 mmol) in tetrahydrofuran (10 mL) was treated with aqueous sodium hydroxide (1.0 N, 0.565 mL, 0.565 mmol). The reaction was stirred overnight at ambient temperature. An additional portion of 1 N sodium hydroxide (0.5 mL, 0.5 mmol) was added and the reaction was stirred an additional 3 h at ambient temperature. The reaction was acidified with aqueous hydrochloric acid (1.0 N, 1.1 mL, 1.1 mmol) and the solvent was evaporated to dryness. The remaining solid was triturated with methanol-ethyl acetate and dried in vacuo to afford 7-(2-Fluoro-4-iodo-phenylamino)-imidazo[1,2-a]pyridine-6-carboxylic acid (150 mg, 100% yield) as a tan-colored solid: ¹H NMR (400 MHz, DMSO-d₆) δ 9.36 (br s, 1H), 8.03 (d, J=2.0 Hz, 1H), 7.85 (dd, J=10.2, 1.7 Hz, 1H), 7.82 (d, J=2.2 Hz, 1H), 7.67 (dm, J=8.5 Hz, 1H), 7.41 (t, J=8.5 Hz, 1H), 6.84 (s, 1H); MS (APCI+) for C₁₄H₉N₃O₂Fl=398 [M+1]. RMECASC IC₅₀=0.027 μM

### EXAMPLE 3

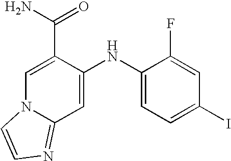

7-(2-Fluoro-4-iodo-phenylamino)-imidazo[1.2-a]pyridine-6-carboxylic acid amide Method A: A solution of 7-(2-Fluoro-4-iodo-phenylamino)-imidazo[1,2-a]pyridine-6-carboxylic acid ethyl ester (172 mg, 0.405 mmol) in ammonia-saturated methanol (10 mL) was heated to 80° C. for 18 h. The cooled reaction mixture was filtered and the tan-colored solid was washed with methanol and dried in vacuo to provide 7-(2-Fluoro-4-iodo-phenylamino)-imidazo[1,2-a]pyridine-6-carboxylic acid amide (86 mg, 54% yield): ¹H NMR (400 MHz, DMSO-d₆) δ 9.56 (d, J=1.7 Hz, 1H), 8.91 (s, 1H), 8.28 (br s, 1H), 7.79 (br s, 1H), 7.73 (br s, 1H), 7.67 (dd, J=10.7, 2.0 Hz, 1H), 7.51 (d brd, J=8.5, 1.2 Hz, 1H), 7.42 (d, J=1.2 Hz, 1H), 7.38 (t, J=8.5 Hz, 1H), 6.90 (s, 1H); ¹⁹F NMR (376 MHz, DMSO-d₆) δ−125.6; MS (APCI+) for C₁₄H₁₀FIN₄O=396.9 [M+1]. C26ELSA IC₅₀=0.0019 μM.

Method B: A solution of 7-(2-Fluoro-4-iodo-phenylamino)-imidazo[1,2-a]pyridine-6-carboxylic acid (120 mg, 0.30 mmol) in dimethylformamide (2 mL) was treated with pyridine (0.027 mL, 0.33 mmol) and pentafluorophenyl trifluoroacetate (0.057 mL, 0.33 mmol). The resultant reaction mixture was stirred overnight at ambient temperature. Concetrated aqueous ammonium hydroxide (7.4 M, 0.28 mL, 2.1 mmol) was added and the reaction was allowed to stand for 3 d. The solvent was evaporated and the residue was chromatographed on silica gel. Elution with ethyl acetate-ethanol-30% ammonium hydroxide (91:8:1) provided 7-(2-Fluoro-4-iodo-phenylamino)-imidazo[1,2-a]pyridine-6-carboxylic acid amide (40 mg, 33%) as a tan-colored solid.

### EXAMPLE 4

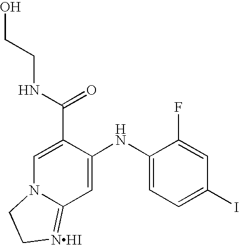 7-(2-Fluoro-4-iodo-phenylamino)-2,3-dihydro-imidazo[1.2-a]pyridine-6-carboxylic acid (2-hydroxy-ethyl)-amide; hydroiodide salt 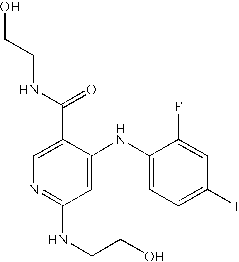
Step A: Preparation of 4-(2-Fluoro-4-iodo-phenylamino)-N-(2-hydroxy-ethyl)-6-(2-hydroxy-ethylamino)-nicotinamide

Ethyl 6-chloro-4-(2-fluoro-4-iodoanilino)nicotinate (1.24 g, 2.95 mmol) and ethanolamine (850 mg, 13.9 mmol) were combined in toluene (20 mL) and heated to reflux for 4 days. The reaction mixture was partitioned between water and ethyl acetate-methanol. The organic layer was washed with water and brine, was dried over magnesium sulfate and was concentrated in vacuo. Upon concentration, 4-(2-Fluoro-4-iodo-phenylamino)-N-(2-hydroxy-ethyl)-6-(2-hydroxy-ethylamino)-nicotinamide crystallized from solution. The crystallization was repeated twice for a total yield of 685 mg (52% yield) of 4-(2-Fluoro-4-iodo-phenylamino)-N-(2-hydroxy-ethyl)-6-(2-hydroxy-ethylamino)-nicotinamide: m. p.>250° C.; ¹H NMR (400 MHz, DMSO-d₆) δ 10.34 (s, 1H), 8.29-8.26 (m, 2H), 7.66 (dd, J=10.5, 2.0 Hz, 1H), 7.48 (d, J=8.0 Hz, 1H), 7.24 (t, J=8.5 Hz, 1H), 6.72 (apparent t, J=5.6 Hz, 1H), 6.01 (s, 1H), 4.67 (apparent q, J=5.8 Hz, 2H), 3.46-3.38 (m, 4H), 3.26-3.19 (m, 4H); ¹⁹F NMR (376 MHz, DMSO-d₆) δ-124.1; MS (APCI+) for C₁₆H₁₈FIN₄O₃=461.0 [M+1].

Step B: Preparation of 7-(2-Fluoro-4-iodo-phenylamino)-2,3-dihydro-imidazo[1.2-a]pyridine-6-carboxylic acid (2-hydroxy-ethyl)-amide: hydroiodide salt

Iodine (180 mg, 0.71 mmol) was added to a solution of 4-(2-Fluoro-4-iodo-phenylamino)-N-(2-hydroxy-ethyl)-6-(2-hydroxy-ethylamino)-nicotinamide (162 mg, 0.35 mmol), triphenylphoshine (200 mg, 0.76 mmol) and triethylamine (0.25 mL, 1.78 mmol) in tetrahydrofuran (8 mL). The reaction mixture was heated with a 60° C. oil bath for 16 h. The solids were filtered and washed with dichloromethane. The solid was further purified by silica gel chromatography. Elution with methanol-dichloromethane (1:4) afforded the hydroiodide salt of 7-(2-Fluoro-4-iodo-phenylamino)-2,3-dihydro-imidazo[1,2-a]pyridine-6-carboxylic acid (2-hydroxy-ethyl)-amide (55 mg, 27% yield) as a tan-colored solid. Recrystallization from methanol afforded analytically pure compound as a monohydrate: m p. 249-251° C.; ¹H NMR (400 MHz, DMSO-d₆) δ 10.54 (s, 1H), 8.67 (t, J=5.6 Hz, 1H), 8.52 (s, 1H), 8.41 (s, 1H), 7.83 (dd, J=9.8, 1.7 Hz, 1H), 7.62 (d, J=8.3 Hz, 1H), 7.22 (t, J=8.3 Hz, 1H), 5.78 (s, 1H), 4.77 (t, J=5.5 Hz, 1H), 4.39 (apparent t, J=8.7 Hz, 2H), 3.77 (apparent t, J=9.2 Hz, 2H), 3.48 (apparent q, J=5.8 Hz, 2H), 3.28 (m, 2H); ¹⁹F NMR (376 MHz, DMSO-d₆) δ-120.1. Anal. Calcd/Found for C₁₆H₁₆FIN₄O₂.HI.H₂O: C, 32.67/33.28; H, 3.26/2.96; N, 9.53/9.45. C26CPA1 IC₅₀=0.177 μM.

### EXAMPLE 5

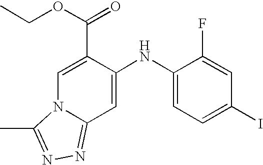 7-(2-Fluoro-4-iodo-phenylamino)-3-methyl-[1,2,4]triazolo[4.3-a]pyridine-6-carboxylic acid ethyl ester
Step A. A solution of ethyl 6-chloro-4-(2-fluoro-4-iodoanilino)nicotinate (1.23 g, 2.92 mmol) in toluene (20 mL) was treated with hydrazine (0.90 mL, 28 mmol) and the resultant reaction mixture was heated to 75° C. for 22 h. The cooled reaction mixture was filtered and the precipitate was washed with toluene (20 mL). The combined filtrate and washings were diluted with ethyl acetate (40 mL) and washed with water (25 mL) and saturated brine (2×25 mL). The combined aqueous was extracted with ethyl acetate (25 mL). The organics were dried over magnesium sulfate and concentrated in vacuo, affording a white solid (0.75 g).
Step B. The solid from step A (0.75 g) was dissolved in triethylorthoacetate (25 mL). Trifluoroacetic acid (0.15 ml, 1.95 mmol) was added and the reaction mixture was heated to 70° C. for three h. The cooled reaction mixture was diluted with ether (50 mL). The yellow precipitate was collected, washed with ether (50 mL), and dried in vacuo affording 7-(2-Fluoro-4-iodo-phenylamino)-3-methyl-[1,2,4]triazolo[4,3-a]pyridine-6-carboxylic acid ethyl ester (0.33 g, 26% yield over 2 steps): m.p. 201-204° C.; ¹H NMR (400 MHz, DMSO-d₆) δ 9.07 (s, 1H), 8.84 (s, 1H), 7.75 (d, J=10.5 Hz, 1H), 7.57 (d, J=8.3 Hz, 1H), 7.39 (t, J=8.5 Hz, 1H), 6.72 (s, 1H), 4.39 (q, J=7.1 Hz, 2H), 2.65 (s, 3H), 1.35 (t, J=7.1 Hz, 3H); ¹⁹F NMR (376 MHz, DMSO-d₆) δ-123.2. Anal. Calcd/Found for C₁₆H₁₄FIN₄O₂: C, 43.66/43.30; H, 3.21/2.95; N, 12.73/12.40; F, 4.32/4.49. RMECASC IC₅₀=3.96 μM.

### EXAMPLE 6

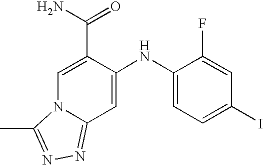 7-(2-Fluoro-4-iodo-phenylamino)-3-methyl-[1,2,4]triazolo[4,3-a]pyridine-6-carboxylic acid amide

A solution of 7-(2-Fluoro-4-iodo-phenylamino)-3-methyl-[1,2,4]triazolo[4,3-a]pyridine-6-carboxylic acid ethyl ester (0.129 g, 0.293 mmol) in ammonia-saturated methanol (5.0 mL) was heated at 80° C. in a sealed tube for 14 h. The reaction mixture was concentrated in vacuo and the yellow solid was chromatographed on silica gel. Elution with methanol-dichloromethane (1:4) provided 7-(2-Fluoro-4-iodo-phenylamino)-3-methyl-[1,2,4]triazolo[4,3-a]pyridine-6-carboxylic acid amide (30 mg, 25% yield). An analytical sample was prepared by trituration with methanol, affording a fine pale-yellow solid: m.p. 300-310° C. (dec); ¹H NMR (400 MHz, DMSO-d₆) δ 9.84 (s, 1H), 8.76 (s, 1H), 8.44 (br s, 1H), 7.99 (br s, 1H), 7.75 (d, J=10.3 Hz, 1H), 7.57 (d, J=8.3 Hz, 1H), 7.44 (t, J=8.5 Hz, 1H), 6.83 (s, 1H), 2.64 (s, 3H); ¹⁹F NMR (376 MHz, DMSO-d₆) δ-124.4; MS(APCI+) 411.9 [M+1]. Anal. Calcd/Found for C₁₄H₁₁FIN₅O: C, 40.90/40.69; H, 2.70/2.50; N, 17.03/16.50.

### EXAMPLE 7

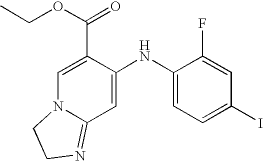 7-(2-Fluoro-4-iodo-phenylamino)-2,3-dihydro-imidazo[1,2-a]pyridine-6-carboxylic acid ethyl ester 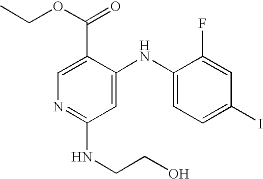
Step A: Preparation of 4-(2-Fluoro-4-iodo-phenylamino)-6-(2-hydroxy-ethylamino)-nicotinic acid ethyl ester
Ethyl 6-chloro-4-(2-fluoro-4-iodoanilino)nicotinate (1.03 g, 2.44 mmol) and ethanolamine (590 mg, 9.67 mmol) were combined in 2-methoxyethanol (15 mL). Concentrated hydrochloric acid (4 drops) was added and the reaction mixture was refluxed for 18 h. The solvent was removed in vacuo and reaction mixture was partitioned between ethyl acetate (75 mL) and water (50 mL). The organic layer was sequentially washed with water, saturated aqueous sodium bicarbonate, water and brine. The organics were dried over magnesium sulfate and concentrated in vacuo. Upon concentration, 6-Chloro-4-(2-fluoro-4-iodo-phenylamino)-N-(2-hydroxy-ethyl)-nicotinamide (510 mg) precipitated from the ethyl acetate solution and was removed by filtration. The filtrate was concentrated in vacuo and chromatographed on silica gel. Elution with methanol-dichloromethane (1:9) afforded 4-(2-Fluoro-4-iodo-phenylamino)-6-(2-hydroxy-ethylamino)-nicotinic acid ethyl ester (90 mg, 8% yield) as a white solid: ¹H NMR (400 MHz, DMSO-d₆) δ 9.39 (br s, 1H), 8.48 (s, 1H), 7.73 (dd, J=10.0, 2.0 Hz, 1H), 7.55 (d, J=8.3 Hz, 1H), 7.28 (t, J=8.3 Hz, 1H), 7.03 (br s, 1H), 5.90 (s, 1H), 4.67 (t, J=5.4 Hz, 1H), 4.23 (q, J=7.1 Hz, 2H), 3.42 (apparent q, J=5.6 Hz, 2H), 3.27 (m partially obscured by HDO, 2H), 1.27 (t, J=7.1, 3H); ¹⁹F NMR (376 MHz, DMSO-d₆) 6-122.8; MS (APCI+) for C₁₆H₁₇FIN₃O₃=446.1 [M+1].
Step B: Preparation of 7-(2-Fluoro-4-iodo-phenylamino)-2.3-dihydro-imidazo[1.2-]pyridine-6-carboxylic acid ethyl ester
A solution of 4-(2-Fluoro-4-iodo-phenylamino)-6-(2-hydroxy-ethylamino)-nicotinic acid ethyl ester (72 mg, 0.16 mmol) in pyridine (3 mL) was treated with p-toluenesulfonyl chloride (40 mg, 0.21 mmol). The resultant solution was stirred 16 h at ambient temperature and 3 h at 60° C. An additional portion of p-toluenesulfonyl chloride (111 mg, 0.58 mmol) was added and heating continued at 60° C. Three h later, another portion of p-toluenesulfonyl chloride (142 mg, 0.74 mmol) was added and heating continued at 60° C. for 18 h. The reaction was quenched with water and the solvent was removed in vacuo. The residue was partitioned between ethyl acetate and water and the organic layer was washed with water, saturated aqueous sodium bicarbonate, water and brine. The combined aqueous washings were extracted with ethyl acetate. The aqueous layer was allowed to stand for 6 days, during which a white precipitate formed. The precipitate was collected by filtration, dissolved in methanol-acetone-ethyl acetate. This extract was dried over magnesium sulfate and concentrated in vacuo affording 7-(2-Fluoro-4-iodo-phenylamino)-2,3-dihydro-imidazo[1,2-a]pyridine-6-carboxylic acid ethyl ester (22 mg, 32% yield): ¹H NMR (400 MHz, DMSO-d₆) δ 8.77 (s, 1H), 7.86 (dd, J=10.0, 2.0 Hz, 1H), 7.66 (J=8.3 Hz, 1H), 7.23 (t, J=8.3 Hz, 1H), 5.65 (s, 1H), 4.40 (apparent t, J=9.0 Hz, 2H), 4.31 (q, J=7.1 Hz, 2H), 3.76 (apparent t, J=9.0 Hz, 2H), 1.30 (t, J=7.1 Hz, 3H); MS (APCI+) for C₁₆H₁₅FIN₃O₂=428.0 [M+1].

### EXAMPLE 8

Cellular Assay for Measuring MEK Inhibition MEK inhibitors were evaluated by determining their ability to inhibit phosphorylation of MAP kinase (ERK) in murine colon 26 (C26) carcinoma cells. Since ERK1 and ERK2 represent the only known substrates for MEK1 and MEK2, the measurement of inhibition of ERK phosphorylation in cells provides direct read out of cellular MEK inhibition by the compounds of the invention. Detection of phosphorylation of ERK was carried out either by Western blot or ELISA format. Briefly, the assays involve treatment of exponentially growing C26 cells with varying concentrations of the test compound (or vehicle control) for one hour at 370 C. For Western blot assay, cells were rinsed free of compound/vehicle and lysed in a solution containing 70 mM NaCl, 50 mM glycerol phosphate, 10 mM HEPES, pH 7.4, 1% Triton X-100, 1 mM Na₃VO₄, 100 μM PMSF, 10 μM leupeptin and 10 μM pepstatin. Supernatants were then subjected to gel electrophoresis and hybridized to a primary antibody recognizing dually phosphorylated ERK1 and ERK2. To evaluate total MAPK levels, blots were subsequently ‘stripped’ and re-probed with a 1:1 mixture of polyclonal antibodies recognizing unphosphorylated ERK1 and ERK2. For pERK ELISA assay, pERK TiterZyme Enzyme immunometric Assay kits were acquired from Assay Designs, Inc (Ann Arbor, Mich.). Briefly, cells were harvested in lysis solution containing 50 mM β-glycerophosphate, 10 mM HEPES, pH7.4, 70 mM NaCl, 2 mM EDTA and 1% SDS and protein lysates were diluted 1:15 with supplied Assay buffer prior to the execution of the assay. The subsequent steps were carried out essentially as recommended by the manufacturer.

The inhibition data generated by the above protocol is disclosed in Table I. If several concentrations of inhibitor were tested, IC₅₀ values (the concentration which gives 50% inhiition) were determined graphically from the dose response curve for % inhibition. Otherwise, percent inhibitions at measured concentrations are reported.

TABLE I
Cellular Inhibition of ERK Phosphorylation by Compounds
of the Invention

| Compound of | C26ELSA IC₅₀ | C26CPA1 IC₅₀ |
|---|---|---|
| Example No. | (μM) | (μM) |
| 3 | 0.0019 | |
| 4 | | 0.177 |

### EXAMPLE 9

Carrageenan-induced Footpad Edema (CFE) Rat Model

Male outbred Wistar rats (135-150 g, Charles River Labs) are dosed orally with 10 mL/kg vehicle or test compound 1 hour prior to administration of a sonicated suspension of carrageenan (1 mg/0.1 mL saline). Carrageenan is injected into the subplantar region of the right hind paw. Paw volume is determined by mercury plethysmography immediately after injection and again five hours after carrageenan injection. Percent inhibition of edema is determined, and the ID40 calculated by linear regression. Differences in swelling compared to control animals are assessed by a 1-way ANOVA, followed by Dunnett's test.

### EXAMPLE 10

Collagen-Induced Arthritis in Mice

Type II collagen-induced arthritis (CIA) in mice is an experimental model of arthritis that has a number of pathologic, immunologic, and genetic features in common with rheumatoid arthritis. The disease is induced by immunization of DBA/1 mice with 100 μg type 11 collagen, which is a major component of joint cartilage, delivered intradermally in Freund's complete adjuvant. The disease susceptibility is regulated by the class 11 MHC gene locus, which is analogous to the association of rheumatoid arthritis with HLA-DR4.

A progressive and inflammatory arthritis develops in the majority of mice immunized, characterized by paw width increases of up to 100%. A test compound is administered to mice in a range of amounts, such as 20, 60, 100, and 200 mg/kg body weight/day. The duration of the test can be several weeks to a few months, such as 40, 60, or 80 days. A clinical scoring index is used to assess disease progression from erythema and edema (stage 1), joint distortion (stage 2), to joint ankylosis (stage 3). The disease is variable in that it can affect one or all paws in an animal, resulting in a total possible score of 12 for each mouse. Histopathology of an arthritic joint reveals synovitis, pannus formation, and cartilage and bone erosions. All mouse strains that are susceptible to CIA are high antibody responders to type 11 collagen, and there is a marked cellular response to CII.

### EXAMPLE 11

SCW-Induced Monoarticular Arthritis

Arthritis is induced as described by Schwab et al., Infection and Immunity, 1991;59:4436-4442 with minor modifications. Rats receive 6 μg sonicated SCW [in 10 μL Dulbecco's PBS (DPBS)] by an intraarticular injection into the right tibiotalar joint on Day 0. On Day 21, the DTH is initiated with 100 μg of SCW (250 μL) administered IV. For oral compound studies, compounds are suspended in vehicle (0.5% hydroxypropyl-methylcellulose/0.2% Tween 80), sonicated, and administered twice daily (10 mL/kg volume) beginning 1 hour prior to reactivation with SCW. Compounds are administered in amounts between 10 and 500 mg/kg body weight/day, such as 20, 30, 60, 100, 200, and 300 mg/kg/day. Edema measurements are obtained by determining the baseline volumes of the sensitized hindpaw before reactivation on Day 21, and comparing them with volumes at subsequent time points such as Day 22, 23, 24, and 25. Paw volume is determined by mercury plethysmography.

### EXAMPLE 12

Mouse Ear-Heart Transplant Model

Fey T. A. et al. describe methods for transplanting split-heart neonatal cardiac grafts into the ear pinna of mice and rats (J. Pharm. and Toxic. Meth., 1998;39:9-17). Compounds are dissolved in solutions containing combinations of absolute ethanol, 0.2% hydroxypropyl methylcellulose in water, propylene glycol, cremophor, and dextrose, or other solvent or suspending vehicle. Mice are dosed orally or intraperitoneally once, twice or three times daily from the day of transplant (Day 0) through Day 13 or until grafts have been rejected. Rats are dosed once, twice, or three times daily from Day 0 through Day 13. Each animal is anesthetized and an incision is made at the base of the recipient ear, cutting only the dorsal epidermis and dermis. The incision is spread open and down to the cartilage parallel to the head, and sufficiently wide to accommodate the appropriate tunneling for a rat or insertion tool for a mouse. A neonatal mouse or rat pup less than 60 hours old is anesthetized and cervically dislocated. The heart is removed from the chest, rinsed with saline, bisected longitudinally with a scalpel, and rinsed with sterile saline. The donor heart fragment is placed into the preformed tunnel with the insertion tool and air or residual fluid is gently expressed from the tunnel with light pressure. No suturing, adhesive bonding, bandaging, or treatment with antibiotics is required.

Implants are examined at 10- to 20-fold magnification with a stereoscopic dissecting microscope without anesthesia. Recipients whose grafts are not visibly beating may be anesthetized and evaluated for the presence of electrical activity using Grass E-2 platinum subdermal pin microelectodes placed either in the pinna or directly into the graft and a tachograph. Implants can be examined 1 to 4 times a day for 10, 20, 30 or more days. The ability of a test compound to ameliorate symptoms of transplant rejection can be compared with a control compound such as cyclosporine, tacrolimus, or orally-administered lefluonomide.

### EXAMPLE 13

The analgesic activity of the compounds of the present invention is assessed by a test with rats. Rats weighing from 175 to 200 g are injected with carrageenan (2% in 0.9% sodium chloride aqueous solution, 100 μL injection volume) into the footpad of one hind limb. The rats are placed on a glass plate with illumination from a halogen lamp placed directly under the injected paw. The time (in seconds) from beginning illumination until the hindlimb was withdrawn from the glass was measured and scored as Paw Withdrawal Latency (PWL). Drug substances were given by oral gavage injection 21/2 hours after carrageenan injection to the footpad. PWL was measured prior to carrageenan injection, just prior to drug injection, and 1, 2 (and sometimes 3) hours after drug injection.

Carrageenan (a polysaccharide extracted from seaweed) causes a sterile inflammation when injected under the skin. Injection into the rat footpad causes little or no spontaneous pain-related behavior but induces hyperalgesia (pain-related behavioral responses of greater intensity than expected) to peripheral thermal or mechanical stimuli. This hyperalgesia is maximal 2 to 3 hours after injection. Treatment of rats with various analgesic drugs reduces hyperalgesia measured in this way and is a conventional test for detection of analgesic activity in rats. (Hargreaves K, Dubner R, Brown F, Flores C, Joris J. A new and sensitive method for measuring thermal nociception in cutaneous hyperalgesia. Pain, 1988;32:77-88 and Kayser V, Guilbaud G. Local and remote modifications of nociceptive sensitivity during carrageenan-induced inflammation in the rat. Pain, 1987;28:99-108). Untreated rats have a PWL of approximately 10 seconds. Carrageenan injection reduces PWL to approximately 3 seconds for at least 4 hours, indicating thermal hyperalgesia. Inhibition of the carrageenan thermal hyperalgesia response is determined by the difference between reduced PWL prior to drug and subsequent to drug treatment, and was expressed as percent inhibition of the response. Administration of MEK inhibitors dose-dependently reduced thermal hyperalgesia.

1. A compound of Formula 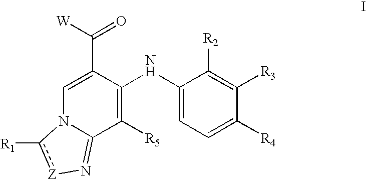
wherein
W is —OH, —O—(CH₂)ₖCH₃, —NH₂, —NH[(CH₂)ₖCH₃], or —NH[O(CH₂)ₖCH₃], wherein the —NH₂ is optionally substituted with between 1 and 2 substituents independently selected from methyl and amino, and the —(CH₂)ₖCH₃ moieties of the —O—(CH₂)ₖCH₃, —NH[(CH₂)ₖCH₃], and —NH[O(CH₂)ₖCH₃] groups are optionally substituted with between 1 and 3 substituents independently selected from hydroxy, amino, alkyl, and cycloalkyl;
Z is N, CH or CH₂;
the dashed line is an optional double bond, wherein the dashed line is a covalent double bond when Z is N or CH, and the dashed line is a covalent single bond when Z is CH₂;
R₁ is hydrogen, C₁₋₆ alkyl, C₂₋₄ alkenyl, C₂₋₄alkynyl, aryl, heteroaryl, or
—(CH₂)ₖO(CH₂)ₖOCH₃, wherein the C₁₋₆ alkyl is optionally substituted with between 1 and 2 substituents independently selected from hydroxy, —COOH, and cyano;
R₂ is hydrogen, chlorine, fluorine or methyl;
R₃ is hydrogen, chlorine, fluorine, methyl, or CF₃;
R₄ is bromine, chlorine, fluorine, iodine, C₁₋₆ alkyl, C₂₋₄ alkenyl, C₂₋₆ alkynyl, C₃₋₆ cycloalkyl, —(CH₂)—C₃₋₆ cycloalkyl, cyano, —O—(C₁₋₄ alkyl), —S—(C₁₋₂ alkyl), —SOCH₃, —SO₂CH₃, —SO₂NR₆R₇, —C≡C—(CH₂)ₙNH₂, —C≡C—(CH₂)ₙNHCH₃, —C≡C—(CH₂)ₙN(CH₃)₂, —C≡C—CH₂OCH₃, —C≡C(CH₂)ₙOH, —C═C—(CH₂)ₙNH₂, —CHCHCH₂OCH₃, —CHCH—(CH₂)ₙNHCH₃, —CHCH—(CH₂)ₙN(CH₃)₂, —(CH₂)ₚCO₂R₆, C(O)C₁₋₃ alkyl, C(O)NHCH₃, —(CH₂)ₘNH₂, —(CH₂)ₘNHCH₃, —(CH₂)ₘN(CH₃)₂, —(CH₂)ₘOR6, —CH₂S(CH₂)ₜ(CH₃), —(CH₂)ₚCF₃, —C—CCF₃, —CH═CHCF₃, —CH₂CHCF₂, —CH═CF₂, —(CF₂)ᵥCF₃, —CH₂(CF₂)ₙCF₃, —(CH₂)ₜCF(CF₃)₂, —CH(CF₃)₂, —CF₂CF(CF₃)₂, or —C(CF₃)₃, wherein the C₁₋₆ alkyl and C₂₋₆ alkynyl are optionally substituted with between 1 and 3 substituents independently selected from hydroxy and alkyl; or R₃ and R₄ can be joined together to form a six-membered aryl ring, five-membered cycloalkyl ring or a five or six-membered heteroaryl ring;
R₅ is hydrogen or fluorine;
R₆ and R₇ are each independently hydrogen, methyl, or ethyl;
k is 0 to 3;
m is 1 to 4;
n is 1 to 2;
p is 0 to 2;
t is 0 to 1;
v is 1 to 5;
and pharmaceutically acceptable salts, C₁₆ amides and C₁₆ esters thereof.

2. The compound of claim 1 wherein W is —OH, —OCH₂CH₃, —NH₂, —NH [(CH₂)₂OH] or —NH[O(CH₂)₂OH].

3. The compound of claim 1 wherein W is —OH, —OCH₂CH₃, —NH₂, —NH [(CH₂)₂OH].

4. The compound of claim 1 wherein R₁ is hydrogen or methyl.

5. The compound of claim 1 wherein R₁ is hydrogen.

6. The compound of claim 1 wherein R₁ is methyl.

7. The compound of claim 1 wherein R₂ is fluorine.

8. The compound of claim 1 wherein R₃ is hydrogen.

9. The compound of claim 1 wherein R₄ is iodine, C₁₋₃ alkyl, C₂₋₄ alkenyl, C₂₋₃ alkynyl, or —S—CH₃.

10. The compound of claim 1 wherein R₄ is iodine.

11. The compound of claim 1 wherein R₄ is iodine, ethyl, allyl or —S—CH₃.

12. The compound of claim 1 wherein R₅ is hydrogen.

13. The compound of claim 1 having a structure which is 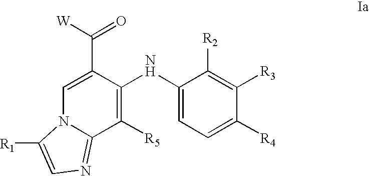
wherein W, R₁, R₂, R₃, R₄, and R₅ are defined as above.

14. The compound of claim 1 having a structure which is 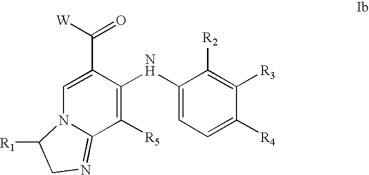
wherein W, R₁, R₂, R₃, R₄, and R₅ are defined as above.

15. The compound of claim 1 having a structure which is 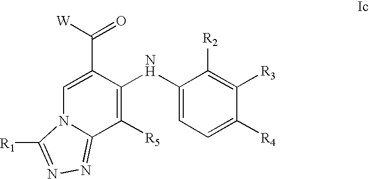
wherein W, R₁, R₂, R₃, R₄, and R₅ are defined as above.

16. The compound of claim 1 which is
7-(2-Fluoro-4-iodo-phenylamino)-imidazo[1,2-a]pyridine-6-carboxylic acid ethyl ester;
7-(2-Fluoro-4-iodo-phenylamino)-imidazo[1,2-a]pyridine-6-carboxylic acid;
7-(2-Fluoro-4-iodo-phenylamino)-imidazo[1,2-a]pyridine-6-carboxylic acid amide;
7-(2-Fluoro-4-iodo-phenylamino)-2,3-dihydro-imidazo[1,2-a]pyridine-6-carboxylic acid (2-hydroxy-ethyl)-amide;
7-(2-Fluoro-4-iodo-phenylamino)-3-methyl-[1,2,4]triazolo[4,3-a]pyridine-6-carboxylic acid ethyl ester;
7-(2-Fluoro-4-iodo-phenylamino)-3-methyl-[1,2,4]triazolo[4,3-a]pyridine-6-carboxylic acid amide; or
7-(2-Fluoro-4-iodo-phenylamino)-2,3-dihydro-imidazo[1,2-a]pyridine-6-carboxylic acid ethyl ester.

17. A pharmaceutical composition comprising a compound of claim 1 and a pharmaceutically acceptable carrier.

18. A method of treating a proliferative disease in a patient in need thereof comprising administering a therapeutically effective amount of a compound of claim 1.

19. A method of treating cancer in a patient in need thereof comprising administering a therapeutically effective amount of a compound of claim 1.

20. A method of treating restonosis, psoriasis, autoimmune disease, atherosclerosis, theumatoid arthritis, heart failure, chronic pain, neuropathic pain, or osteoarthritis in a patient in need thereof comprising administering a therapeutically effective amount of a compound of claim 1.

21. A method of treating cancer in need thereof comprising administering a therapeutically effective amount of a compound of claim 1 in combination with radiation therapy or at least one chemotherapeutic agent.
